(19) United States
(12) Patent Application Publication
(10) Pub. No.: US 2014/0205609 A1
(43) Pub. Date: Jul. 24, 2014

(54) METHODS FOR INDUCING SYSTEMIC IMMUNE RESPONSES TO CANCER

(71) Applicant: Fred T. Valentine, New York, NY (US)

(72) Inventor: Fred T. Valentine, New York, NY (US)

(21) Appl. No.: 14/161,840

(22) Filed: Jan. 23, 2014

Related U.S. Application Data
(60) Provisional application No. 61/756,094, filed on Jan. 24, 2013.

(51) Int. Cl. A61K 39/00

(52) U.S. Cl. CPC A61K 39/0011
USPC 424/142.1; 424/277.1

(57) ABSTRACT

Pharmaceutical compositions comprising molecules produced by antigen-stimulated peripheral blood mononuclear cells (PBMCs) that induce systemic immune responses to cancers are encompassed herein, as are methods for making and administering such molecules and pharmaceutical compositions comprising same. Also encompassed herein are mixtures of molecules produced by antigen-stimulated PBMCs and compositions thereof for use in treating cancer. Methods for stimulating a systemic immune response in a subject afflicted with a cancer are also envisioned, whereby molecules produced by antigen-stimulated PBMCs or supernatants comprising same are administered to the subject, wherein the molecules stimulate an immune response to the cancer in the subject.

### CROSS REFERENCE TO RELATED APPLICATION

This application claims priority under 35 USC §119(e) from U.S. Provisional Application Ser. No. 61/756,094, filed Jan. 24, 2013, which application is herein specifically incorporated by reference in its entirety.

### GOVERNMENTAL SUPPORT

The research leading to the present invention was supported, at least in part, by National Institutes of Health Grant No. R01 CA 19529 and P01 CA 16247. Accordingly, the Government has certain rights in the invention.

### FIELD OF THE INVENTION

Pharmaceutical compositions comprising molecules produced by antigen-stimulated peripheral blood mononuclear cells (PBMCs) that induce systemic immune responses to cancers are encompassed herein, as are methods for making and administering such pharmaceutical compositions. Melanoma is an exemplary tumor type for which such pharmaceutical compositions would confer benefit to patients. Also encompassed herein are methods for stimulating an immune response in a subject afflicted with a cancer, whereby molecules produced by antigen-stimulated PBMCs or supernatants comprising same are administered to the subject, wherein the molecules or supernatants comprising same stimulate an immune response to the cancer in the subject.

### BACKGROUND OF THE INVENTION

The citation of references herein shall not be construed as an admission that such is prior art to the present invention.

The current status of metastatic malignant melanoma is as follows: approximately 8,000 individuals die from malignant melanoma per year in the United States according to the American Cancer Society. The median overall survival for patients with stage 4 metastatic melanoma has been 8 to 10 months, with 2 to 7% alive at 5 years^{1,2}. Although surgery and radiation therapy are used in the treatment of metastatic disease, systemic therapy is considered the mainstay for treatment. Traditional single-agent chemotherapy is well tolerated, but confers response rates of only 5-20%, with no complete remissions and no significant prolongation of survival.

Long term durable complete remissions of metastatic malignant melanoma also have not been regularly observed in spite of recent advances in the treatment of patients with a BRAF inhibitor, vemurafenib, resulting in very significant and dramatic responses for 7 months to one year, and occasional complete responses, in the approximately 50% of patients whose malignancy has a mutated version of the BRAF signaling protein³⁴. Unfortunately, resistant melanoma is rapidly selected with additional mutations in the previously susceptible protein that bypass the block in signaling, and might also be facilitated by the continued presence of the drug. Moreover, in the phase III study, 38% of participants required modification of dose due to toxicities. A second recently licensed treatment for metastatic disease uses an immunologic approach in which CTLA4, a molecule that inhibits immune responses, is blocked by an antagonistic monoclonal antibody against CTLA4, ipilimumab, thereby enhancing immune responses that may be present. In a phase III trial of stage 3c and 4 patients, the median overall survival was 10 months with an overall survival rate of 23.5% of patients at 24 months^{2,5}. The median progression free survival was 2.86 months with a survival rate of 10% at 48 months, and only an overall 1.5% rate of complete responses. Because ipilimumab enhances all ongoing immune responses, serious and occasionally lethal adverse events may occur (23% of recipients had grade 3 or 4 adverse events). More recently antagonistic monoclonal antibodies directed against the immunological inhibitory molecule and ligand PD-1 or PDI-1 increased survival in patients with metastatic melanoma by enhancing all ongoing immune responses^{6 7}. Autologous CD8 T cells selected for reactivity to a melanoma antigen, expanded ex vivo, and reinfused back into the patients have also resulted in partial responses^{8,9}. However, achieving long term durable complete remissions remains challenging.

### SUMMARY OF THE INVENTION

The invention relates generally to methods and agents for inducing an effective immune response to a cancer. The invention further relates to methods and agents for treating a cancer patient (e.g., a patient with melanoma). More particularly, methods and agents for inducing an effective immune response to melanoma in a subject are described herein. Immune responses so induced have been shown to confer complete regression of disease in patients with advanced melanoma, whereby patients remained disease free for decades after treatment. In brief, the present inventor has shown that intralesional injection of autologous cytokines or intracutaneous injection of cytokines with irradiated autologous melanoma cells induces the development of systemic cellular immune responses against melanoma, as evidenced by lymphocytic infiltrates and frequent complete regressions of never injected metastatic nodules. The infiltrating lymphocytes are primarily CD8 T cells and are able to kill autologous, but not allogeneic melanoma cells ex vivo. Complete regressions are frequent and of surprising durability, with a significant number of patients surviving disease-free for 5 to 29 years after entry with stage 3c or 4 disease. Other than mild transient local tenderness in injected and occasional noninjected nodules, no adverse events have been observed.

In addition to inducing systemic immune responses against metastatic cancers that result in complete and durable regressions of metastases, the cytokine method as described herein can be used to induce tumor-specific immune responses concurrently with or prior to treatment with Ipilimumab, Nivolumab or other therapies designed to block the normal physiological restraints on immune responses. These therapies are immunologically nonspecific in that they enhance all ongoing immune responses in the patient and therefore can be associated with serious adverse events, including autoimmune diseases and perforations of the intestine. Initial treatment or concurrent treatment with the cytokine method will induce robust immune responses specific for the patient's tumor. This should permit much lower doses and shorter durations of treatment with Ipilimumab, Nivolumab and similar therapies, thereby minimizing the adverse events associated with these therapies. Most importantly, the initial or concurrently use of methods described herein should greatly increase the frequency of complete regressions of metastatic disease, which occur infrequently with Ipilimumab or Nivolumab.

In accordance with the present findings, a method for inducing a systemic immune response to a metastatic cancer in a subject is presented, the method comprising administering supernatants from activated peripheral blood mononuclear cells (PBMCs) to the subject, wherein the supernatants are administered intratumorally and repeatedly.

In an embodiment thereof, the metastatic cancer is a melanoma, breast cancer, renal cancer, or lung cancer. In a particular embodiment, the metastatic cancer is a tumor of the skin (e.g., a melanoma).

In yet another embodiment, the supernatants are administered in a small volume. In a more particular embodiment, the small volume is about 0.05 to 3.0 or about 0.05 to 2.5 milliliters per injected metastatic nodule. In an even more particular embodiment, the small volume is about 0.1 to 2 milliliters or about 0.05 to 1.0 milliliters per injected metastatic nodule.

In a particular embodiment, the supernatants are generated by contacting peripheral blood mononuclear cells (PBMCs) in vitro with an antigen recognized by the PBMCs, thereby activating the PBMCs. In a more particular embodiment, the peripheral blood mononuclear cells (PBMCs) are isolated from the subject and the supernatants are generated by contacting the PBMCs in vitro with an antigen recognized by the PBMCs, thereby activating the PBMCs. The antigen may, for example, be a microbial antigen. Microbial antigens suitable for such purposes include, without limitation, tuberculin PPD skin test antigen, streptokinase for injection, Candida albicans skin test antigen, or tetanus toxoid suitable for booster use. In a particular aspect, the antigen can be identified and/or confirmed empirically as an antigen recognized by the PBMCs and capable of activating the PBMCs. In a particular embodiment, the supernatants are administered at a frequency of once or twice per week or once every 2 to 4 weeks. In a more particular embodiment the supernatants are administered at a frequency of once per week or once per 2 weeks. It will, however, be readily apparent that supernatants can be administered at intervals of every day, every other day, bi-weekly, weekly, or once every two, three, or four weeks. The precise pattern for supernatant administration may be determined by a skilled practitioner attending the subject or patient.

In another embodiment, the subject in need thereof is a mammalian subject. In a more particular embodiment, the mammalian subject is a human.

In another aspect, a method for inducing a systemic immune response to a metastatic cancer in a subject is presented, the method comprising administering irradiated autologous tumor cells and supernatants from activated peripheral blood mononuclear cells (PBMCs) to the subject, wherein the irradiated autologus tumor cells and supernatants are administered intradermally and repeatedly. In an embodiment thereof, the metastatic cancer is a melanoma, breast cancer, renal cancer, or lung cancer. In a more particular embodiment, the metastatic cancer is a tumor of the skin (e.g., a melanoma).

In another embodiment, the irradiated autologous tumor cells and supernatants are administered in a small volume. In a more particular embodiment, the small volume is about 0.05 to 3.0 or about 0.05 to 2.5 milliliters per injected metastatic nodule. In an even more particular embodiment, the small volume is about 0.1 to 2 milliliters or about 0.05 to 1.0 milliliters per injected metastatic nodule.

Activated supernatants are generated in accordance with methods described herein. Activated supernatants may, for example, be generated by stimulating isolated autologous peripheral blood mononuclear cells (PBMC) at about one million cells/ml in 10% autologous plasma-containing tissue culture medium (e.g., modified Eagles medium) for 18 to 36 hours. While autologous PBMCs are employed for convenience and to avoid potential exposure of the recipient to infectious agents, no immunological reason requires the use of autologous cytokines, and the same composition of cytokines could be obtained from alternative sources. In a particular embodiment, they are stimulated for 18-24 hours. Isolated PBMCs are stimulated with sterile microbial antigens to which the donor's PBMC are known to respond. Microbial antigen (about 1 to 10 μg/ml) may be used to stimulate the patient's PBMC in a test tube to proliferate and to release cytokines. Supernatants may also be prepared by briefly pulsing PBMCs with antigen.

In another embodiment, the supernatants are generated by contacting peripheral blood mononuclear cells (PBMCs) in vitro with an antigen recognized by the PBMCs, thereby activating the PBMCs. In a more particular embodiment, the peripheral blood mononuclear cells (PBMCs) are isolated from the subject and the supernatants are generated by contacting the PBMCs in vitro with an antigen recognized by the PBMCs, thereby activating the PBMCs. The antigen may, for example, be a microbial antigen. Microbial antigens suitable for such purposes include, without limitation, tuberculin PPD skin test antigen, streptokinase for injection, Candida albicans skin test antigen, or tetanus toxoid suitable for booster use. In a particular aspect, the antigen can be identified and/or confirmed empirically as an antigen recognized by the PBMCs and capable of activating the PBMCs.

In a particular embodiment, the irradiated autologous tumor cells and activated PBMC supernatants are administered at a frequency of once per week or once per 2 weeks or once per 2 to 4 weeks. Irradiated autologous tumor cells may be mixed with activated PBMC supernatants and injected or co-injected at the same intradermal site. In a more particular embodiment, the irradiated autologous tumor cells are generated by isolating tumor cells from the subject and irradiating the tumor cells in vitro with 20,000 rads (200 Gy) using a standard blood bank irradiator. Autologous tumor cells may be isolated from a subject in advance of treatment and stored at the temperature of liquid nitrogen in keeping with standard practice until needed. In circumstances wherein the patient has no residual detectable disease (e.g., is in remission), methods described herein are utilized in an adjuvant setting to delay or prevent reappearance of disease. Accordingly, under such circumstances, tumor cells are isolated from the subject during an active disease state and stored, so as to provide a stock of autologous tumor cells for potential future use.

In another embodiment, the subject in need thereof is a mammalian subject. In a more particular embodiment, the mammalian subject is a human.

Other objects and advantages will become apparent to those skilled in the art from a review of the ensuing detailed description, which proceeds with reference to the following methods and the attendant claims.

### DETAILED DESCRIPTION

Methods and agents for inducing an effective immune response to a metastatic cancer are described herein. The invention further relates to methods and agents for treating a patient afflicted with a metastatic cancer, such as metastatic melanoma. More particularly, methods and agents for inducing an effective immune response to melanoma in a subject are described herein. Immune responses induced using methods and agents described herein have been shown to confer complete disease regression in patients with advanced metastatic melanoma, such that patients have remained disease free for decades following treatment.

More particularly, the present inventor has shown that intralesional injection of autologous cytokines or intracutaneous injection of cytokines with irradiated autologous melanoma cells induces the development of systemic cellular immune responses directed against the melanoma cells. Lymphocytic infiltrates and complete regressions of both injected and never injected metastatic nodules reflect the systemic nature of the anti-melanoma cellular immune responses induced. It is, moreover, noteworthy that the infiltrating lymphocytes are primarily CD8 T cells and are able to kill autologous, but not allogeneic, melanoma cells ex vivo. Complete regressions are frequent and surprisingly durable, with a significant number of patients diagnosed with stage 3c or 4 diseases surviving disease-free for 5 to 29 years after onset of treatment. No significant adverse events have been associated with treatment regimens described herein.

It is noteworthy that although this survival data was generated in an immunological rather than in a therapeutic study, the clinical outcomes following the injections of autologous cytokines compare favorably with the outcomes achieved with ipilimumab that were evaluated in a slightly more advanced cohort of patients, which resulted in a median overall survival of 10 months (cytokines 27 months); a rate of overall survival of 14% at two years (cytokines 54% at 2 years and 29% at 5 years). The most conspicuous difference was in the significantly higher frequency of complete regressions of all metastases in recipients of cytokines. Indeed, 20% of patients receiving cytokines had a complete regression and no evident disease at 5 years as compared to ipilimumab recipients experiencing complete regressions at any time, which was only 0.5% of patients. In the cytokine recipients, the most advanced patents (stage IV) had a median overall survival of 20 months, with 15% of these stage IV patients remaining free of all metastases for a median of 276 months (23 years). In the absence of therapy, stage IV patients have a median survival of 11 months^{1,2}. The methods and uses described herein have, therefore, been proven to be therapeutically effective for the treatment of cancer in humans.

In a further embodiment, the cytokine method as described herein can be used to induce tumor-specific immune responses prior to treatment with Ipilimumab⁵, Nivolumab⁶ or other therapies⁷ designed to block the normal physiological restraints on immune responses. These therapies are immunologically nonspecific in that they enhance all ongoing immune responses in the patient and therefore, can be associated with serious adverse events, including autoimmune diseases and perforations of the intestine. Initial treatment with the cytokine method will induce robust immune responses specific for the patient's tumor. This should permit much lower doses and shorter durations of treatment with Ipilimumab, Nivolumab and similar therapies, thereby minimizing the adverse events associated with these therapies. See also Vonderheide et al. (Nature Med. 2013; 19:1098-1100), which is incorporated herein in its entirety. Most importantly, the initial use of the cytokine method should greatly increase the frequency of complete regressions of metastatic disease, which occur infrequently with Ipilimumab or Nivolumab.

Ipilimumab⁵ is a monoclonal antibody that binds to and blocks the biolgical activity of CTLA4, a molecule on activated T lymphocytes that normally nonspecifically restrains all ongoing immune responses. When used to treat metastatic melanoma the monoclonal antibody (3 mg/kg) is administered intravenously every 3 weeks for 4 infusions. Tumor responses usually are observed after 14 weeks. 23% of recipients have grade 3 or 4 (serious) adverse events, and complete regressions are seen in only 3% of patients⁵. Additional particulars relating to therapeutic regimens involving ipilimumab are described in Hodi et al. (New England Journal of Medicine 2010; 363:711-23), Wolchok et al. (New England Journal of Medicine 2013; 369:122-133), and in ClinicalTrials.gov numbers NCT00094653 and NCT01024231, the entire content of each of which is incorporated herein by reference.

Nivolumab⁶ is a monoclonal antibody blocking the activity of a different molecule on activated T cells, PD-1, which also normally nonspecifically inhibits ongoing immune responses. It is administered intravenously at 1 or 3 mg/kg every 2 weeks for multiple cycles, and tumor responses are not seen for many weeks. 14% of recipients experienced grade 3 or 4 adverse events, and complete responses occurred in only 1% of melanoma patients⁶. Additional particulars relating to therapeutic regimens involving nivolumab are described in Topalian et al. (New England Journal of Medicine 2012; 366:2443-54), Wolchok et al. (New England Journal of Medicine 2013; 369:122-133), and in ClinicalTrials.gov numbers NCT00730639 and NCT01024231, the entire content of each of which is incorporated herein by reference.

A second monoclonal antibody against PD-1, Lambrolizumab⁷, is administered intravenously at 10 mg/kg every 2 or 3 weeks for up to one year. 13% of patients had grade 3 or 4 adverse events, and 5% complete responses. For all 3 of these monoclonal antibodies occasional adverse events were lethal and others required steroids or other immunosuppressive therapies. Additional particulars relating to therapeutic regimens involving lambrolizumab are described in Brahmer et al. (New England Journal of Medicine 2012; 366:2455-65), Hamid et al. (New England Journal of Medicine 2013; 369:134-144), and in ClinicalTrials.gov number NCT01295827, the entire content of each of which is incorporated herein by reference.

As described in this application, intralesional cytokine injections by themselves induce systemic tumor-specific cellular immune responses resulting in a high proportion of durable complete regressions. This cytokine treatment prior to the administration of any of the monoclonal antibodies just outlined should significantly shorten the required duration of infusion of the monoclonal antibodies, decrease the concentrations of the antibodies administered down to one tenth the current doses, decrease the frequency of adverse events, and most importantly result in greatly increased numbers of complete regressions. The potential to administer the cytokines and the monoclonal antibodies simultaneously together must be investigated.

As described in this application the injection of cytokines and irradiated autologous tumor cells also induces systemic tumor-specific cellular immune responses. The injection of cytokines and irradiated autologous tumor cells as described prior to the infusion of the monoclonal antibodies also should decrease the concentrations of the antibodies administered down to one tenth the current doses, decrease the frequency of adverse events, and most importantly result in greatly increased numbers of complete regressions. The potential to administer the cytokines, irradiated tumor cells and the monoclonal antibodies together must be investigated.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. Terminology

As used herein, the term “immunomodulator” refers to an agent which is able to modulate an immune response. An example of such modulation is an enhancement of cell activation or of antibody production.

The term “effective amount” of an immunomodulator refers to an amount of an immunomodulator sufficient to enhance an immune response, be it cell-mediated, humoral or antibody-mediated. In accordance with the methods described herein, an effective amount of an intratumorally or subcutaneously injected immunomodulator can be in the range of about 1-10,000 picograms, 1-1,000 picograms, 1-100 picograms, 1-20 picograms, or 1-10 picograms for a human subject. In a particular embodiment, an intratumorally or subcutaneously injected immunomodulator is in the range of 20-10,000 picograms. In the context of the instant methods, it is understood that the supernatants from activated PBMCs comprise a combination of individual immunomodulators present in the above amounts and thus, methods described herein relate to effects of the immunomodulators in combination.

The exact effective amount necessary will vary from subject to subject, depending on the species, age and general condition of the subject, the severity of the condition being treated, the mode of administration, etc. Thus, it is not possible to specify an exact effective amount. However, the appropriate effective amount may be determined by one of ordinary skill in the art using only routine experimentation or prior knowledge in the vaccine art.

An “immunological response” to an antigen in a vaccine or initiated by an immunomodulator is the development in the host of a cellular- and/or antibody-mediated immune response to the antigen or antigenic cancer of interest. Usually, such a response consists of the subject producing antibodies, B cells, helper T cells, suppressor T cells, and/or cytotoxic T cells directed specifically to an antigen or antigens. In a particular embodiment, antigens are presented on a cancer cell/s.

The term “comprise” is generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The term “isolated” as used herein may be used to refer to the state in which peptides or proteins described herein, or nucleic acids encoding same are used. Peptides/proteins and nucleic acids will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Peptides/proteins and nucleic acids may be formulated with diluents or adjuvants and still for practical purposes be isolated—for example the peptides/proteins will normally be mixed with gelatin or other carriers if used to coat microtiter plates for use in immunoassays, or will be mixed with pharmaceutically acceptable carriers or diluents when used in diagnosis or therapy. The term may also be used with respect to cells that are derived from a subject or patient. In certain embodiments, cells that are derived/isolated from a subject or patient are cultured, treated and/or purified in vitro following separation from the patient.

As used herein, “pg” means picogram, “ng” means nanogram, “ug” or “μg” mean microgram, “mg” means milligram, “ul” or “μl” mean microliter, “ml” means milliliter, “I” means liter.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. Amino acids are referred to herein using standard polypeptide nomenclature.

A “replicon” is any genetic element (e.g., plasmid, chromosome, virus) that functions as an autonomous unit of DNA replication in vivo; i.e., capable of replication under its own control.

A “vector” is a replicon, such as a plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

As used herein, the terms “restriction endonucleases” and “restriction enzymes” refer to bacterial enzymes which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding a cytokine or cytokines expressed by activated PBMCs or comprising or consisting of sequences which are degenerate thereto. DNA sequences having the nucleic acid sequence encoding the peptides of the invention are contemplated, including degenerate sequences thereof encoding the same, or a conserved or substantially similar, amino acid sequence. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC or GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCC or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in the sequences encoding the proteins or peptides generated by activated PBMCs, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at Ph 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups: Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine CO and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues, preferably at least about 80%, and most preferably at least about 90, 91, 92, 93, 94, 95, 96, 97, 98, or 99% of the amino acid residues are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The term ‘agent’ may be used to refer to any molecule, including activated PBMC-derived peptides or other polypeptides, antibodies, polynucleotides, chemical compounds and small molecules. In particular the term agent includes compounds such as test compounds or drug candidate compounds. The term ‘modulator agent” as used herein refers to an agent whose presence alters an interaction (e.g., a biochemical or physical interaction) relative to a control or inert agent. A modulator agent may, therefore, increase/enhance or decrease/reduce such an interaction relative to a control or inert agent. In a particular aspect, a modulator agent may enhance a systemic immune response to a metastatic cancer in a subject and is, therefore, identified as an enhancer, promoter, or inducer of same.

The term ‘agonist’ refers to a ligand that stimulates the receptor to which the ligand binds in the broadest sense or stimulates a response that would be elicited on binding of a natural ligand to a binding site.

The term ‘assay’ means any process used to measure a specific property of a compound or agent. A ‘screening assay’ means a process used to characterize or select compounds based upon their activity from a collection of compounds.

“Preventing” or “prevention” refers to a reduction in risk of acquiring a disease or disorder.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

The term ‘treating’ or ‘treatment’ of any disease or infection refers, in one embodiment, to ameliorating the disease or infection (e.g., arresting the disease or growth of a causative infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease.

The phrase “pharmaceutically acceptable” refers to molecular entities and compositions that are physiologically tolerable and do not typically produce an allergic or similar untoward reaction, such as gastric upset, dizziness and the like, when administered to a human.

As used herein, the term “autologous” refers to organs, tissues, cells, or proteins isolated from a donor patient that are later re-introduced into the donor patient. Accordingly, the donor and recipient are the same patient in autologous transfers. The term “autologous PBMC”, for example, refers to PBMCs that have been isolated from a subject and then administered to the same patient. Typically, and in accordance with the present methods, isolated PBMCs may be isolated from a patient and stimulated in cell culture and activated supernatants generated thereby administered to the patient. The term “autologous tumor cells”, for example, refers to tumor cells that have been isolated from a subject and then administered to the same patient. Typically, and in accordance with the present methods, the isolated tumor cells are irradiated prior to administration to the patient.

The term “irradiated” describes methods and processes whereby tumor cells are rendered incapable of dividing. In a particular embodiment, single cell suspensions may be obtained from the visceral metastases of subjects with metastatic melanoma by gentle mechanical teasing, and brief collagenase/protease/DNAse digestion. The cells are viably frozen, and aliquots of 10⁷ autologous tumor cells irradiated with 20,000 rads (200 Gy), mixed with the cytokines and injected intradermally every 1 to 2 weeks.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” is any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); and (xii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contain the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct anti-cancer or anti-tumor specific antibody. The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF) or a toxin (e.g., ricin) or anti-mitotic or apoptotic agent or factor.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

### B. Further Aspects of the Detailed Description

At the outset of the experiments described herein, the present inventor postulated that although metastatic melanoma rarely provokes an effective immune response, melanoma cells may present target epitopes for cytotoxic CD8 cells (CTLs) should an immune response be initiated. One of the earliest steps in the adaptive immune response is the release of multiple cytokines, which at physiologic concentrations act in concert to have antigen nonspecific effects on adjacent cells, to attract, activate and expand local CD4 and CD8 T lymphocytes, affect antigen presenting cells, potentially modulate the expression of antigens on melanoma or other cancer cells and/or initiate an immune response to adjacent (melanoma or other tumor) antigens. The approach described herein is based, at least in part, on two immunological concepts. The first relates to the injection of a mixture of cytokines. In this regard, it is noteworthy that normal immune responses typically involve multiple cytokines. The families of cytokines and chemokines now probably exceed 70 different identified molecules, and probably others yet to be described. The second relates to the use of low concentrations of cytokines that induce immunity without causing significant side effects. Accordingly, the low concentrations of cytokines used in the accordance with the present methods are injected at low levels sufficient to induce immunity to the tumor cells, but insufficient to attack tumor cells directly.

To test this hypothesis in vivo, the present inventor initiated an Institutional Review Board (IRB) approved immunological study in patients by bringing antigen (melanoma cells), cytokines and circulating mononuclear cells together by injecting autologous cytokines weekly into some metastatic nodules, while other nodules, often at some distance, in that patient were never injected, in order to detect the development of systemic immune responses. Intriguingly, both injected and never injected metastatic nodules developed dense lymphocytic infiltrates following such injections. Regressions of both injected and non-injected metastatic nodules, and complete regressions in some patients who remained free of evident disease for many months were documented. Although a number of patients who participated in these studies and were free of detectable disease when last seen have been lost to follow up, further efforts to follow up with others who participated in the study revealed that several patients have survived free of disease for over 10 to 20 years since receiving injections of autologous cytokines. This is particularly surprising given that these patients were diagnosed with advanced metastatic disease, as described in greater detail below, before onset of treatment.

In summary, the findings presented herein demonstrate that injection of mixtures of autologous cytokines into metastatic lesions (intralesional injection) or intracutaneous injection of a combination of mixtures of autologous cytokines with irradiated autologous melanoma cells results in the development of lymphocytic infiltrates and associated regressions of non-injected nodules, and in many but not all patients, complete and often durable regressions. The median overall survival of 27 months, with 20% of patients free of evident disease 5 years after entering the study and a median disease-free survival in this group of 14 years, compare favorably with the therapeutic benefits of current licensed treatments. The improved nature of the present methods is apparent, even when taking into consideration the likelihood that some participants in the licensing trials may have had more advanced disease relative to those treated using methods described herein.

The findings of the present inventor are even more noteworthy given the generally disappointing results observed in various therapeutic regimens involving administration of cytokines to melanoma patients. As discussed in greater detail herein below, high dose IL-2 and types 1 and 2 interferon administered as anti-tumor agents have, for example, induced occasional responses but were usually associated with severe toxicities. As a consequence, these cytokines now are seldom used for melanoma due to the combination of relatively infrequent durable responses and pronounced side effects.

The term cytokines refers to a large group of regulatory polypeptide molecules, which along with chemokines, are coordinately secreted, primarily by activated cells of the innate and adaptive immune systems, and interact in concert to initiate, modulate and regulate immune responses. Type one interferons, for example, are produced by many types of cells in the body as an antiviral response. Cytokines, formerly called soluble mediators of cellular immunity, were initially identified and isolated by their actions in vitro on cells¹⁰⁻¹⁴. A number of cytokine molecules have been purified and recombinant proteins prepared by molecular biology techniques. However, it is now appreciated that different cytokine molecules often have overlapping biological activities^{15,16}, and that the biological effect of a given cytokine in vivo may be dependent on or influenced by the concomitant presence of other cytokines acting on the cell^{17,18}. A cytokine such as IL-2 that facilitates the proliferation of lymphocytes may, for example, cause the stimulated lymphocytes to die, and also to enhance the activity of inhibitory T regulatory cells^{15,19,20}. The activity of a cytokine also depends on the level and specificity of the receptors currently expressed on cells on which it is acting¹⁵, and activity of one cytokine may stimulate the release of a different cytokine from cells¹⁸. As an additional complexity, studies with genetically manipulated mice incapable of making a given cytokine (knock out mice) suggest that multiple (6+) cytokines and additional factors yet to be identified may be required to induce and maintain cytotoxic CD8 T cells. The majority of cytokines act at the cell to cell interface and are present in vivo at exceedingly low concentrations.

A mixture of cytokines and chemokines governs the attraction of lymphocytes, their retention at the site of an immune response, the activation of dendritic cells, antigen presentation to lymphocytes, the proliferative expansion of T cell clones, interactions between CD4 and CD8 T cells, and the down regulation of an immune response. The net effect of cytokines on an immune response results from the interactions of multiple cytokines, and cannot be accurately predicted by a list of activities of the individual molecules involved. Indeed, in keeping with the present invention, the mixture of cytokines itself is the biological and potential therapeutic reagent.

In contrast, previous and current experimental immunological approaches for the treatment of cancer have used high dose-limiting concentrations of several recombinant cytokines. These approaches to the immunotherapy of metastatic melanoma, with occasional studies of renal or lung cancer are summarized in the following paragraphs. High dose [2.8 mg administered intravenously (i.v.) every 8 hours] Interleukin-2 (IL-2) administered as a single cytokine to patients with advanced metasatic melanoma induces some level of tumor regression in 6 to 15% of recipients, only occasionally of long lasting duration, and uncommon complete regressions²¹. When combined with an MHC-matched peptide melanoma vaccine, suitable for use only in patients with a specific histocompatibility type, high dose IL-2, induced a 9% complete response and 7% partial response, a progression free survival of 2.2 months, as compared with 1% CR and 5% PR and a progression free survival of 1.6 months for IL-2 alone. Median overall survivals were 17.8 months with vaccine and 11.1 months for IL-2 alone. However, grade 3 to 5 adverse events were seen in all IL-2 recipients, requiring management in an intensive care unit.²¹ Due to these severe toxicities, IL-2 is seldom used for treatment of metastatic melanoma, even though it is licensed for that purpose. It is noteworthy that lower doses of IL-2 (one tenth the high dose level) either without or with GMCSF have not proven to be clinically effective.²²

Type 1 interferons (primarily IFNα2) at maximally tolerated doses (20 million units/day) have been evaluated for the treatment of advanced metastatic melanoma with minimal clinical responses of short duration observed and a 25% withdrawal rate due to toxicities²³. As adjuvant therapy for patients whose metastases have been surgically resected, however, high dose IFNα2 results in a decreased rate of recurrence, and in 2 of 4 studies has demonstrated a prolongation of survival, although 25% of subjects withdrew due to toxicities²³. IFNα2 has been licensed as adjuvant therapy for this type of patient.

Studies involving GMCSF have sought to elicit immune responses against metastatic melanoma by systemic injection as adjunctive treatment after resection (200 μg/day)²⁴, and by injecting the cytokine intra-lesionally in a vaccinia or adeno²⁵, or oncolytic herpes virus²⁶ vector expressing the cytokine. These studies have resulted in limited prolonged disease free survival, and in a phase II trial a 26% objective response (16% CR) with a few CR lasting a number of months. Eighty-five percent of recipients, however, experienced fever, aches, fatigue and other flu-like symptoms. DNA encoding IL-12 as a plasmid or in a canarypox vector induced the expression of detectable IL 12 in injected metastases and an occasional transient regression^{27,28}. The intralesional injection of canarypox vectors expressing both IL-12 and the costimulatory molecule B7.1 into 12 patients with metastatic melanoma, however, had no beneficial effects on the disease²⁹.

Combinations of two recombinant cytokines have also been evaluated as potential therapies for metastatic cancers. Combinations of high dose or low dose ( 1/10^{th} of high dose) IL-2 with tumor necrosis factor (TNF) were no more effective than IL-2 alone and were associated with severe dose limiting toxicities^{30,31}. IL-2 combined with IFN-β has induced some partial responses in metastatic cancer in one but not all studies, and has been associated with severe toxicities^{32,33}. Combinations of IL-12 with low to moderate doses of IL-2 resulted in no significant tumor responses³⁴. Intralesional injections of IL-2 and GMCSF in metastatic melanoma resulted in 12% partial responses³⁵. A randomized study of the systemic administration of low doses of IL-2 with or without GMCSF revealed no clinical benefit in either arm²², however alternating low and high dose IL-2 combined with high dose GMCSF and chemotherapy resulted in a 15% complete response rate of limited duration in one study³⁶. Combinations of GMCSF with IL-4 or IL-6 were also ineffective^{37,38}, as were combinations of IL-2 and IL-4³⁹ and IL-2 and IFN-θ^{40,41} Large randomized studies of IL-2 combined with IFNα at maximum doses demonstrated rare complete responses and an 8% partial response rate associated with severe toxicities^{42,43}, and the investigators found no justification for the use of this combination.

Combinations of 3 recombinant cytokines at maximally tolerated doses have also been evaluated. IL-2, IFN-α and TNF-α⁴⁴; and IL-2, IFN-γ and GMCSF⁴⁵ induced minimal clinical responses in renal cancers and were no better than dual cytokines. The combination of dose limiting concentrations of IL-2, GMCSF and IFNα in metastatic melanoma resulted in one transient partial response, but severe toxicities.⁴⁶ A lower dose of IL-2, combined with IFN-α and GMCSF resulted in a 5% complete response rate in renal cell carcinoma, as effective as high dose IL-2 but with reduced toxicity⁴⁷.

The very limited clinical benefits and severe toxicities demonstrated in these studies of high doses of single or combination cytokines result from the use of only a small number of cytokines selected primarily on the basis of their activities in vitro or in vivo at super-physiological concentrations, and underestimate the complexities of cytokine interactions in vivo. In vivo, a much larger number of cytokines act in concert at exceedingly low doses to induce and regulate new immune responses, as outlined herein above. The toxic, dose-limiting doses of individual recombinant cytokines used in the studies just cited are 1 million to 500 million times larger than the doses of the same cytokine present in the cytokine mixture used in the present methods. In addition, the cytokine mixture used in this invention contains over 30 cytokines, rather than the 1 to 3 recombinant cytokines used to date in attempts to treat human cancers. In short, the present invention differs conceptually, methodologically, and in clinical efficacy from previous attempts at the immunotherapy of human cancers.

### Preparation of Molecules Produced by Stimulated PBMCs

Cytokine mixtures are prepared by stimulating autologous peripheral blood mononuclear cells (PBMC) one million cells/ml in 10% autologous plasma-containing tissue culture medium (modified Eagles medium) for 20 to 24 hours. PBMC are prepared from fresh peripheral heparinized blood by centrifugation over Ficol-Hypaque⁴⁸. The cells are washed twice, placed in culture, and then stimulated with sterile microbial antigens (commercially available) to which the patient's PBMC respond as assessed by previous proliferation assays. For example, if a patient had been infected with Candida or with M. tuberculosis in the past, they would have a positive skin test for that antigen. The same concentration of the microbial antigen (1 to 10 μg/ml) will stimulate the patient's PBMC in a test tube to proliferate and to release cytokines. Commercially available microbial antigens that may be used include, without limitation: tuberculin PPD skin test antigen, streptokinase for injection, candida albicans skin test antigen, and tetanus toxoid for booster use. Alternatively, the cytokines can be prepared by pulsing the cells briefly (1 hour) with the antigen, washing the cells, and collecting the supernatant medium after 24 hours. Cytokines are prepared in a biosafety, filtered air cabinet using strict sterile technique, and are passed through a 0.2 micron filter before injection. The cytokine mixtures contain a variable range of exceeding small concentrations of cytokines (5 to 5,000 picograms/ml of each cytokine in the mixture). Supernatants prepared in parallel by briefly pulsing the PBMCs with antigen contained only 5 to 40% of these levels, but contained no detectable microbial antigen. In a small number of subjects, supernatants prepared in this manner induced complete regressions, although these supernatants were not as effective as those with larger concentrations of cytokines (and some residual antigen). Supernatants from PBMC pulsed with an antigen to which the donor is not sensitive do not stimulate the PBMC of a donor exquisitely sensitive to a different antigen and thus, can be viewed as control supernatants. A cytokine mixture generated following activation of PBMC as described herein is injected in a volume of 0.05 to 0.2 ml into cutaneous or subcutaneous metastatic nodules via a #27 needle, very much like performing a skin test. A given patient with multiple metastatic nodules may receive a total volume of, for example, 1 to 2 ml during one session, wherein multiple metastatic nodules are injected.

It will be appreciated that cytokine mixtures useful in methods described herein may be prepared by stimulating autologous PBMC at a range of different cell concentrations, from approximately 100,000 to about ten million cells/ml. It will also be appreciated that plasma tissue culture medium other than modified Eagle medium may be used in the preparation of cytokine mixtures. Examples of suitable plasma tissue culture media include, without limitation: RPMI 1640, and various chemically-defined, protein-free, and serum-free formulations such as PFHM II. Medium may contain 15%, 10%, 1% or zero serum or plasma. It will be understood that variations in the above protocol are also envisioned and known in the art and the above protocol is presented for illustrative purposes only and is not intended to be limiting.

In accordance with the methods described herein, isolated PBMCs may be activated in vitro using cell culture systems as described herein or by following routine protocols understood in the art. Supernatants of activated PBMCs are then administered to a subject/patient in need thereof using techniques known in the art such as intratumoral or intradermal injection, local injection in the vicinity of a tumor, and/or local infusion via pump or a similar device in the tumor or vicinity of the tumor.

More specifically with regard to preparation of supernatants from activated PBMCs, a mixture of molecules containing cytokines and chemokines may be prepared by stimulating PBMCs with antigen (antigen-stimulated PBMCs) in autologous plasma-containing tissue culture medium for 20-24 hours. Such a mixture may be referred to herein as antigen-stimulated PBMC supernatants or activated PBMC supernatants. In a particular embodiment, PBMCs are stimulated with sterile microbial antigens (commercially available from a variety of vendors) to which the patient's PBMCs have been shown to respond, as determined by performing, for example, a proliferation assay. Accordingly, PBMCs are exquisitely sensitive and specific to particular antigens. A proliferation assay may be conducted by culturing 100,000 PBMCs in replicate wells containing different microbial antigens, and assessing the level of proliferation by adding tritiated thymidine for 6 hours on day 6 of culture and quantifying the amount of DNA synthesis in response to the antigenic stimulation. Proliferation assays may be performed via other protocols, which are known in the art and a matter of routine practice. Microbial antigens known to be adequate include: PPD Tuberculin PPD for skin testing, solution 5 TU/0.1 ml, (Tubersol) Sanofi Pasteur, Swiftwater, Pa. 18370; Tetanus toxoid for booster immunization. Solution standardized to contain 4 Lf/0.5 ml., Sanofi Pasteur, Swiftwater, Pa. 18370; Candida albicans skin test antigen, solution for injection. Extract of culture, sterile in buffered saline pH 8.0 containing 0.03% USP human albumin. Standardized to give 5 to 10 mm induration in a delayed hypersensitivity skin test at 48 hours in human donors of known sensitivity (Candin) Allermed Laboratories, Inc San Diego, Calif. 92111; Streptokinase for injection sterile powder in vials of 250,000 units (Streptase). No longer manufactured in the US. Available from ZLB Behring Marburg, Germany.

Each lot of antigen is standardized to give the same level of a proliferative response as the previous lot in an initial parallel experiment. Concentrations of each antigen should stimulate a proliferative response of PBMC from a sensitive donor to a stimulation index of at least a 10 fold increase in thymidine incorporation. In general by weight this requires about 1 to 10 micrograms of antigen/ml of culture fluid. The same concentration of a microbial antigen shown to induce proliferation may subsequently be used to stimulate the patient's PBMCs in vitro (e.g., in a test tube, tissue culture well, or the like) at a concentration of 100,000 to one million PBMC/ml to release the aforementioned mixture of molecules including cytokines and chemokines. Commercially available microbial antigens that may be used to stimulate PBMCs include, without limitation: tuberculin PPD skin test antigen, streptokinase for injection, Candida albicans skin test antigen, and tetanus toxoid suitable for booster use. Alternatively, antigen-stimulated PBMC supernatants can be prepared by pulsing the cells briefly (1 to 2 hours) with the desired antigen, washing the cells, and collecting the supernatant medium after 24 hours. Cytokines are prepared in a biosafety, filtered air cabinet using strict sterile technique, and are passed through a 0.2 micron filter before injection. The mixtures contain a range of exceedingly small concentrations (picograms to nanograms/ml) of individual cytokines.

Supernatants prepared in parallel by briefly pulsing the PBMCs with antigen and culturing for 24 hours contain approximately 5 to 40% of the cytokine levels achieved following 20-24 hours of antigen stimulation, but contain no detectable microbial antigen. As a control experiment to evaluate whether the process of pulsing cells for up to 2 hours with an antigen eliminates detectable antigen in the supernatants obtained after 24 hours of culture, PBMC from a donor not sensitive to a given antigen are pulsed with that antigen, and the supernatants taken at 24 hours are added to PBMC from a donor exquisitely sensitive to that antigen. If the sensitive donor's cells do not respond, then no detectable antigen was present in the pulsed supernatants. Supernatants from activated PBMCs comprise a mixture of cytokines and chemokines, wherein each of the cytokines is present at a low concentration (ranging typically from 20-5000 picograms/ml).

The cytokine mixture is then administered to a patient in need thereof via injection in a volume of 0.05 to 0.2 ml into cutaneous or subcutaneous metastatic nodules using, for example, a #27 needle. The procedure is performed in similar fashion to that of a skin test. At weekly or every other weekly visits, a patient with multiple metastases may receive up to a total of 2 mls.

In an alternative embodiment, irradiated autologous melanoma cells are prepared from surgically excised visceral or cutaneous metastases (after sending a portion of the specimen for pathological examination and storage). Single cell suspensions are prepared using sterile techniques, washed, and viably frozen in autologous serum, placed in sealed vials and stored in liquid nitrogen vapor. Prior to injection, a vial of 10⁷ cells is thawed, washed once, irradiated with 20,000 rads (200 Gy), mixed with 0.2 to 0.3 ml of autologous cytokines and injected intradermally every two weeks, rotating between sites draining to different lymph node areas. The cells may also be used as targets for assessing the ability of PBMCs from a patient to kill autologous tumor cells ex vivo.

For patients who do not have cutaneous or subcutaneous metastases that can be injected, but for whom autologous tumor cells are available from surgical excisions of metastatic disease, single cell suspensions can be prepared, irradiated and mixed with small quantities of autologous cytokines and then injected intradermally as described herein above with respect to autologous melanoma cells. Inaccessible metastases can be followed by clinical and radiographic methods. If the patient does not have clinically detectable disease then irradiated melanoma cells and cytokines will be used in an adjuvant setting.

Accordingly, methods and agents for inducing an effective immune response to tumors, particularly those of the skin (e.g., melanoma), are presented herein. In one aspect, a method directed to promoting the appearance of infiltrating lymphocytes (primarily CD8 T cells) capable of killing cancer cells, including melanoma cells, in the subject is envisioned. The infiltrating lymphocytes may be further assessed ex vivo by evaluating their composition with regard to cell types and their killing specificity. Based on the findings of the present inventor, infiltrating lymphocytes generated as described herein demonstrate specificity with respect to killing in that they can kill autologous, but not allogeneic cancer cells ex vivo.

Accordingly, cytokine mixtures described herein have application and use, alone or in combination with irradiated autologous tumor cells, or other immune system modulators, T cell modulators, antibodies, vaccines, antigens, or chemotherapeutics for stimulating, facilitating or enhancing desired immune system or immune cell actions or activities, particularly those directed against tumors, in promoting tumor regression and/or improved patient survival. In addition to directly resulting in complete regressions, the injection of cytokine mixtures by methods described herein may be used to induce and increase specific anti-tumor immune responses concurrently or before the subsequent administration of Ipilimumab, Nivolumab or related molecules to permit the use of less toxic levels of these enhancers of immune responses, and to obtain more frequent complete regressions of metastases.

The present invention also includes cytokines or functional fragments thereof in mixtures, or agents or other drugs determined to possess the same activity, which are covalently attached to or otherwise associated with other molecules or agents. These other molecules or agents include, but are not limited to, molecules (including antibodies or antibody fragments) with distinct recognition, targeting or binding characteristics, immune cell modulators, immune cell antigens, toxins, ligands, adjuvants, and chemotherapeutic agents.

Peptides and proteins described herein may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to peptides or proteins described herein using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, and urease. Labels further include chemical moieties such as biotin which may be detected via binding to a specific cognate detectable moiety, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Proteins and peptides of and for use in the present invention may include synthetic, recombinant or peptidomimetic entitites. The peptides may be monomers, polymers, multimers, dendrimers, concatamers of various forms known or contemplated in the art, and may be so modified or multimerized so as to improve activity, specificity or stability. For instance, and not by way of limitation, several strategies have been pursued in efforts to increase the effectiveness of antimicrobial peptides including dendrimers and altered amino acids (Tam et al (2002) Eur J Biochem 269 (3): 923-932; Janiszewska et al (2003) Bioorg Med Chem Lett 13 (21):3711-3713; Ghadiri et al. (2004) Nature 369(6478):301-304; DeGrado et al (2003) Protein Science 12(4):647-665; Tew et al. (2002) PNAS 99(8):5110-5114; Janiszewska et al (2003) Bioorg Med Chem Lett 13 (21): 3711-3713). U.S. Pat. No. 5,229,490 discloses a particular polymeric construction formed by the binding of multiple antigens to a dendritic core or backbone.

Conjugates or fusion proteins of the present invention, wherein cytokines or functional fragments thereof as described herein are conjugated or attached to other molecules or agents further include, but are not limited to binding members conjugated to a cell targeting agent or sequence, chemical ablation agent, toxin, immunomodulator, another cytokine, cytotoxic agent, chemotherapeutic agent or drug.

In vitro assays are described herein which may be utilized by the skilled artisan to further or additionally screen, assess, and/or verify the activities of cytokines or functional fragments thereof as described herein, including further assessing immune responses targeted against tumor cells. Cell based assays and in vitro methods are described herein and were utilized to perform experiments as described, for example, in the Examples.

In vivo animal models of human cancers and immune response thereto, and in vivo animal models reconstituted with a human immune system may be utilized by the skilled artisan to further or additionally screen, assess, and/or verify the activity of mixtures of cytokines or functional fragments thereof as described herein, including further assessing immune response targeted against tumor cells in vivo. Such animal models include, but are not limited to, models of immune system modulation or immune response.

Proteins, peptides, immune activators or agents described herein (e.g., mixtures of cytokines or functional fragments thereof) may be administered to a patient in need of treatment via any suitable route, including by intratumoral, intradermal, subcutaneous, intravenous, intraperitoneal, intramuscular injection, or via oral, rectal, buccal or intranasal administration. The precise dose will depend upon a number of factors, including whether the proteins, peptides, immune activators or agents are for treatment, for adjuvant therapy after all detectable tumor has been surgically excised or decreased below detection by chemotherapy, or for prevention. In an adjuvant setting, for example, if all detectable metastases have been excised, cytokine mixtures in combination with irradiated autologous melanoma cells can be administered so as to prevent or delay recurrence of disease. The dosage or dosing regime of an adult patient may be proportionally adjusted for children and infants, and also adjusted for other administration or other formats, in proportion for example to molecular weight or immune response. Administration or treatments may be repeated at appropriate intervals, at the discretion of the physician.

Proteins, peptides, immune activators or agents described herein are generally administered in the form of a pharmaceutical composition, which may comprise at least one component in addition to the proteins, peptides, immune activators or agents. Pharmaceutical compositions according to the present invention, and for use in accordance with the present invention, may comprise, in addition to active ingredient, a pharmaceutically acceptable excipient, carrier, buffer, stabiliser or other materials known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

A composition may be administered alone or in combination with other treatments, therapeutics or agents, either simultaneously or sequentially dependent upon the condition to be treated. In addition, the present invention contemplates and includes compositions comprising the proteins, peptides, immune activators or agents herein described and other agents or therapeutics such as immune modulators, antibodies, immune cell stimulators, or adjuvants. The composition may also be administered with, or may include combinations along with immune cell antigen antibodies or immune cell modulators.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A protein, peptide, immune activator or agent can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Accordingly, also encompassed herein is a mixture of cytokines or functional fragments thereof as described herein or nucleic acid sequences encoding same or compositions thereof further comprising a pharmaceutically acceptable buffer, for use in treating a patient with a tumor or tumors, such as melanoma, wherein said composition reduces tumor burden and/or alleviates symptoms of the tumor in the patient when administered to the patient in a therapeutically effective amount. Such compositions may also have utility for use in prophylaxis for a patient in an adjuvant setting, wherein no detectable disease is present, but wherein the patient is at risk for the reappearance of detectable disease, wherein said composition prevents or alleviates symptoms in the patient when administered to the patient in an effective amount. Also encompassed herein is the use of a therapeutically effective amount of a mixture of cytokines or functional fragments thereof as described herein or nucleic acid sequences encoding same or compositions thereof further comprising pharmaceutically acceptable buffers in the manufacture of a medicament for treating a patient with a tumor, such as melanoma, wherein the medicament alleviates or prevents symptoms of the tumor when administered to the patient. Also encompassed herein is a mixture of cytokines or functional fragments thereof as described herein or nucleic acid sequences encoding same and compositions thereof for use in treating cancer (e.g., melanoma) in a subject.

The peptide or agent containing compositions are, for example, conventionally administered intratumorally, intradermally, intramuscularly, intravenously, as by injection of a unit dose, or orally. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient/s, and degree of activation and immune response desired. Precise amounts of active ingredient required to be administered depend on the judgment of the practitioner and are peculiar to each individual. Suitable regimens for initial administration and follow on administration are also variable, and may include an initial administration followed by repeated doses at appropriate intervals by a subsequent injection or other administration.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

### Nucleic Acids

The present invention further provides an isolated nucleic acid encoding a protein, peptide, immune activator or agent of the present invention. Nucleic acid includes DNA and RNA. In a preferred aspect, the present invention provides a nucleic acid which codes for a polypeptide as described herein.

The present invention also provides constructs in the form of plasmids, vectors, and transcription or expression cassettes which comprise at least one polynucleotide. The present invention also provides a recombinant host cell which comprises one or more of such constructs. A nucleic acid encoding any protein or peptide described herein forms an aspect of the present invention, as does a method of production of the protein or peptide which method comprises expression from encoding nucleic acid therefor. Expression may conveniently be achieved by culturing recombinant host cells containing the nucleic acid under appropriate conditions. Following production by expression, a protein or peptide may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, and many others. A common, preferred bacterial host is E. coli. The expression of antibodies and antibody fragments in prokaryotic cells such as E. coli is well established in the art.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing a nucleic acid as described herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above.

Another feature of this invention is the expression of DNA sequences contemplated herein, particularly those encoding the cytokines or functional fragments thereof described herein. As is well known in the art, DNA sequences may be expressed by operatively linking them to an expression control sequence in an appropriate expression vector and employing that expression vector to transform an appropriate unicellular host. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences (sequences that control the expression of a DNA sequence operatively linked to it) may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, RI.I, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products. Considering these and other factors a person skilled in the art will be able to construct a variety of vector/expression control sequence/host combinations that will express the DNA sequences of this invention on fermentation or in large scale animal culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Introduction

As shown herein, the present inventor has shown that intralesional injection of autologous cytokines or intracutaneous injection of cytokines with irradiated autologous melanoma cells promotes systemic cellular immune responses directed against the melanoma cells. Lymphocytic infiltrates and complete regressions of both injected and never injected metastatic nodules reflect the systemic nature of the anti-melanoma cellular immune responses induced. Infiltrating lymphocytes are primarily CD8+ T cells and are able to kill autologous melanoma cells ex vivo. The specificity of the cell killing is evident in that the CD8+ T cells do not kill allogeneic melanoma cells. Complete regressions are frequent and surprisingly durable, with a significant number of patients diagnosed with stage 3c or 4 disease surviving disease-free for 5 to 23 years after onset of treatment. No significant adverse events have been associated with treatment regimens described herein.

Patients and Methods:

On entry 89 patients had clinically palpable cutaneous/subcutaneous in transit metastases of an extremity or multiple metastases on the trunk or head (equivalent to stage IIIC), and some had distant cutaneous metastases (stage IV). Because the goals of this study were immunological rather than an evaluation of clinical outcome, detailed staging including scans was not routinely performed. However patients with clinically evident visceral metastases at the time of entry were excluded. Clinical data is abstracted from 89 patients with metastatic melanoma, advanced stage 3 or 4, who participated in different aspects of these studies. All had developed 2 to over 100 metastases (mean of 21) during the 2 months prior to entry, and many had failed to respond to earlier chemotherapy. In keeping with the experimental design, the present inventor prepared a mixture of autologous cytokines by stimulating PBMC in vitro for 24 hours with a microbial antigen to which the patient responded, or by pulsing the cells briefly with the antigen, and collecting the supernatant medium. Cytokine containing supernatants were collected after a short stimulus in order to avoid the potential for subsequent inhibitory factors since robust ongoing immune responses are physiologically shut down. Supernatants prepared in parallel by briefly pulsing the PBMCs with antigen contained approximately 25 to 40% of the levels present in supernatants from PBMCs stimulated by antigen for 24 hours, but contained no detectable microbial antigen, and have been shown to initiate complete regressions in a small number of patients.

Two Methods of Administration of Autologous Cytokines have been Evaluated.

For the majority of participants with cutaneous or subcutaneous metastatic disease as described above, cytokines were injected weekly into some metastatic nodules, while other nodules, distal or proximal to the injected nodules, and occasionally on the contralateral side of the body, were never injected. After about 12 weeks some nodules began to regress over 2 to 10 weeks; usually injected nodules regressed first but in some patients never injected nodules regressed first. When injected nodules in a patient regressed completely, never-injected nodules also regressed completely. Both non-injected and injected nodules in patients who only received cytokines from cells pulsed with antigen regressed completely. Nodules continued to regress over several months. If minimal metastatic disease was initially present, 67% of patients experienced complete regressions of all nodules. Never injected nodules on the contralateral side have regressed. However, new nodules frequently appeared even while non-injected nodules were regressing, suggesting the possibility of antigenic variation and escape or of other intrinsic differences between nodules in the same patient. The newly developing nodules on occasion would regress completely if some of them were injected. However, the persistent appearance of new nodules in spite of ongoing regressions accounted for the majority of failures to achieve durable no evident disease (NED) status.

A second category of patients presented with a visceral metastasis and no identified primary (1 patient) or developed visceral or distant nodal metastases while subcutaneous nodules were regressing (4 patients). In a separate IRB-approved protocol, single cell suspensions of cells were obtained from the visceral metastases of these individuals, viably frozen, and aliquots of 10⁷ autologous tumor cells irradiated with 20,000 rads (200 Gy) mixed with their autologous cytokines and injected intradermally every 2 weeks. Three of the 5 patients remained free of disease for 18 to 23 years. The use of cytokines with irradiated autologous tumor cells in patients at high risk of advanced recurrent metastases represents the use of this strategy in an adjuvant setting. When mixed with the cytokines, autologous tumor cells appear to be sufficiently antigenic such that a range of 10,000 to 100 million tumor cells per injection induces antitumor immune responses. In addition, this strategy can be used regularly when residual visceral or cutaneous metastases are still present.

The durability of regressions and survival data from the combined modes of administration are incomplete since some of these patients have not been followed for years, and some who were in complete remission have been lost to follow up. The survival data on 89 participants is calculated only until death, start of chemotherapy, or the last time they were examined. Median overall survival was 27 months; entry to death, chemo, or last known NED, and the mean overall survival 64 months. After entering the study, 29% of patients lived for 5 or more years with or without evident disease (overall survival).

After entering the study, 54% of subjects lived over 2 years, 43% lived over 3 years, and 29% lived for 5 or more years with or without evident disease (overall survival). 36% of subjects had complete regressions with no evident disease (NED) for a period of 2 months or more. Twenty three patients (26%) remained without evident disease at their most recent follow up (range 4 to 348 months), with a median duration of 119 months and a mean of 163 months (14 years) after entering this study. 20% of patients entering this program are known to have been free of detectable disease for at least 5 years after entering the study, with 13% being followed for at least 10 years free of disease (median 23 yrs; range 10 to 29 yrs), including a few patients who initially presented with stage IV disease. The median survival of only those patients who progressed after entering the study with stage 3c or 4 metastatic disease was 18 months and the mean 30 months to death or to receiving chemotherapy. This suggests that in addition to inducing long term complete regressions, the immunotherapy may have prolonged survival in patients with progressive disease. Median overall survival from entry into the study until death, chemotherapy or last date known to be NED was 27 months, and the mean overall survival 64 months.

It should be noted that although this survival data was generated in an immunological rather than in a therapeutic study, the outcomes following the injections of autologous cytokines compare favorably to outcomes achieved with ipilimumab, which result in a median overall survival of 10 months (cytokines 27 months); a rate of overall survival of 14% at two years (cytokines 54% at 2 years and 29% at 5 years); ipilimumab rate of complete remission at any time 0.5% (cytokines 20% complete remission and no evident disease at 5 years.)

Safety:

Depigmented areas developed at the site of regressed nodules in some patients. One patient experienced transient local urticaria at the site of injection, which did not recur. Local transient (persisting 1 to 3 days) erythema and tenderness of injected and never injected nodules in a significant minority of patients, and occasional increased fatigue for a day were the only other side effects observed.

Histology:

Microscopic examination of regressing, never injected nodules revealed a dense lymphocytic infiltrate, restricted to the tumor, composed of CD8 and in many patients also CD4 T cells as assessed by immunohistology and by flow cytometry of single-cell suspensions. Prior to the injection of cytokines, lymphocytic infiltrates are not observed.

Cytotoxic Function of Infiltrating Cells:

In several subjects for whom fresh tumor cells and tumor infiltrating lymphocytes (TIL) were available, the present inventor was able to document that the TIL kill autologous but not allogeneic fresh tumor cells in vitro as assessed by Cr⁵¹ release. Similarly, in 5 patients with regressing nodules blood lymphocytes were shown to be able to kill autologous fresh melanoma cells in vitro. Cytotoxicity was eliminated by depletion of CD8 cells, but not by depletion of NK cells. Melanoma cell lines are not used. In subjects injected repeatedly with irradiated autologous melanoma cells mixed with autologous cytokines an increase in circulating blood lymphocytes cytotoxic to autologous melanoma cells in vitro has been documented.

This invention may be embodied in other forms or carried out in other ways without departing from the spirit or essential characteristics thereof. The present disclosure is therefore to be considered as in all aspects illustrate and not restrictive, the scope of the invention being indicated by the appended claims, and all changes which come within the meaning and range of equivalency are intended to be embraced therein.

Various references are cited throughout this Specification, each of which is incorporated herein by reference in its entirety.

### REFERENCES

1. Ascierto P A, Grimaldi A M, Curti B, et al. Future perspectives in melanoma research. Meeting report from the “Melanoma research: a bridge from Naples to the World. Napoli, Dec. 5-6 2011”. Journal of translational medicine 2012; 10:83.
2. Robert C, Thomas L, Bondarenko I, et al. Ipilimumab plus dacarbazine for previously untreated metastatic melanoma. The New England journal of medicine 2011; 364:2517-26.
3. Chapman P B, Hauschild A, Robert C, et al. Improved survival with vemurafenib in melanoma with BRAF V600E mutation. The New England journal of medicine 2011; 364:2507-16.
4. Alcala A M, Flaherty K T. BRAF inhibitors for the treatment of metastatic melanoma: clinical trials and mechanisms of resistance. Clinical cancer research: an official journal of the American Association for Cancer Research 2012; 18:33-9.
5. Hodi F S, O'Day S J, McDermott D F, et al. Improved survival with ipilimumab in patients with metastatic melanoma. The New England journal of medicine 2010; 363:711-23.
6. Topalian S L, Hodi F S, Brahmer J R, et al. Safety, activity, and immune correlates of anti-PD-1 antibody in cancer. The New England journal of medicine 2012; 366:2443-54.
7. Brahmer J R, Tykodi S S, Chow L Q, et al. Safety and activity of anti-PD-L1 antibody in patients with advanced cancer. The New England journal of medicine 2012; 366:2455-65.
8. Yee C T, J. A. Byrd, D. Adoptive T cell therapy using antigen-specific CD8+ T cell clones for the treatment of patients with metastatic melanoma: In vivo persistence, migration, and antitumor effect of transferred T cells. Proc Natl Acad Sci USA 2002; 99:16168-73.
9. Butler M O, Friedlander P, Milstein M I, et al. Establishment of antitumor memory in humans using in vitro-educated CD8+ T cells. Sci Transl Med 2011; 3:80ra34.
10. Valentine F T, David J R. Soluble mediators of delayed hypersensitivity. Progress in Immunology First International Conference of Immunology. Washington, D.C. USA: Academic Press, New York; 1971:1527-32.
11. Bloom B R, Bennett B. Mechanism of a reaction in vitro associated with delayed-type hypersensitivity. Science 1966; 153:80-2.
12. Valentine F T, Lawrence H S. Lymphocyte stimulation: transfer of cellular hypersensitivity to antigen in vitro. Science 1969; 165:1014-6.
13. Shadduck R K, Nunna N G, Boggs D R,. Granulocyte colony stimulating factor. 3. Effect of alkylating agent induced granulocytopenia. Proc Soc Exp Biol Med 1971; 137:1479-82.
14. Valentine F T. Soluble factors produced by lymphocytes. Annals of the New York Academy of Sciences 1974; 221:317-23.
15. Lin J X, Migone T S, Tsang M, et al. The role of shared receptor motifs and common Stat proteins in the generation of cytokine pleiotropy and redundancy by IL-2, IL-4, IL-7, IL-13, and IL-15. Immunity 1995; 2:331.
16. Prlic M, Lefrancois L, Jameson S C,. Multiple choices: regulation of memory CD8 T cell generation and homeostasis by interleukin (IL)-7 and IL-15. J Exp Med 2002.; 195:F49-52.
17. Fleischman W, Georgiades J, Osborne L. Potentiation of interferon activity by mixed preparations of fibroblast and immune interferon. Infect Immun 1979; 26:248-153.
18. Hamilton S E, Prlic M, Jameson S C. Environmental conservation: bystander CD4 T cells keep CD8 memories fresh. Nat Immunol 2004; 5:873-4.
19. Malek T, Bayer A. Tolerance, not immunity, crucially depends on IL-2. Nat Rev Immunol 2004; 4:665-74.
20. Shrikant P, Mescher M F. Opposing effects of IL-2 in tumor immunotherapy: promoting CD8 T cell growth and inducing apoptosis. Journal of immunology (Baltimore, Md.: 1950) 2002; 169:1753-9.
21. Schwartzentruber D J, Lawson D H, Richards J M, et al. gp100 peptide vaccine and interleukin-2 in patients with advanced melanoma. The New England journal of medicine 2011; 364:2119-27.
22. Smith J, Kurt R, Baher A. Immune effects of escalating doses of granulocyte-macrophage colony-stimulating factor added to a fixed, low-dose, inpatient interleukin-2 regimen: a randomized phase I trial in patients with metastatic melanoma and renal cell carcinoma. Journal of Immunotherapy 2003; 26:130-8.
23. Tarhini A, Gogas H, Kirkwood J. IFN-a in the Treatment of Melanoma. Journal of immunology (Baltimore, Md.: 1950) 2012; 189:3789-93.
24. Spitler L, Grossbard M, Ernstoff M, et al. Adjuvant Therapy of Stage III and IV Malignant Melanoma Using Granulocyte-Macrophage Colony-Stimulating Factor. Journal of clinical oncology: official journal of the American Society of Clinical Oncology 2000; 18:1614-21.
25. Dranoff G. GM-CSF-secreting melanoma vaccines. Oncogene 2003; 22:3188-92.
26. Senzer N N, Kaufman H L, Amatruda T, et al. Phase II clinical trial of a granulocyte-macrophage colony-stimulating factor-encoding, second-generation oncolytic herpesvirus in patients with unresectable metastatic melanoma. Journal of clinical oncology: official journal of the American Society of Clinical Oncology 2009; 27:5763-71.
27. Heinzerling L, Burg G, Dummer R,. Intratumoral injection of DNA encoding human interleukin 12 into patients with metastatic melanoma: clinical efficacy. Hum Gene Ther 2005; 16:35-48
28. Cocco C, Pistoia V, Airoldi I. New perspectives for melanoma immunotherapy: role of IL-12. Curr Mol Med 2009; 9:459-69.
29. Triozzi P, Allen K, Carlisle R, Craig M, LoBuglio A, Conry R. Phase I Study of the Intratumoral Administration of Recombinant Canarypox Viruses Expressing B7.1 and Interleukin12 in Patients with Metastatic Melanoma Clinical cancer research: an official journal of the American Association for Cancer Research 2005; 11:4168-75.
30. Krigel R L, Padavic-Shaller K, Toomey C, Comis R L, Weiner L M. Phase I study of sequentially administered recombinant tumor necrosis factor and recombinant interleukin-2. Journal of immunotherapy with emphasis on tumor immunology: official journal of the Society for Biological Therapy 1995; 17:161-70.
31. Yang S, Grimm E, Parkinson D. Clinical and Immunomodulatory Effects of Combination Immunotherapy with Low-Dose Interleukin 2 and Tumor Necrosis Factor alpha in Patients with Advanced Non-Small Cell Lung Cancer: A Phase I Trial. Cancer Research 1991; 51:3669-76.
32. Krigel R L, Padavic-Shaller K A, Rudolph A R, Konrad M, Bradley E C, Comis R L. Renal cell carcinoma: treatment with recombinant interleukin-2 plus beta-interferon. Journal of clinical oncology: official journal of the American Society of Clinical Oncology 1990; 8:460-7.
33. Duggan D, Santarelli M, Zamkoff K. A phase ii study of recombinant interleukin-2 with or without recombinant interferon-beta in non-Hodgkin's lymphoma. a study of the cancer and leukemia group B. Journal of immunotherapy (Hagerstown, Md.: 1997) 1992; 12:115-22.
34. Gollob J, Veenstra K, Parker R. Phase I trial of concurrent twice-weekly recombinant human interleukin-12 plus low-dose IL-2 in patients with melanoma or renal cell cancer. Journal of clinical oncology: official journal of the American Society of Clinical Oncology 2003; 21:2564-73.
35. Ridolfi L, Ridolfi R, Ascari-Raccagni A. Intralesional GM-CSF followed by subcutaneous IL2 in metastatic melanoma. Journal of the European Academy of Dermatology and Venereology 2001; 15:218-23.
36. O'Day S, Boasberg P, Piro L. Maintenance biotherapy for metastatic melanoma with interleukin-2 and granulocyte macrophage-colony stimulating factor improves survival for patients responding to induction concurrent biochemotherapy. Clinical cancer research: an official journal of the American Association for Cancer Research 2002; 8:2775-81.
37. Gitlitz B, Figlin R, Kiertscher S, Moldawer N, Rosen F, Roth J. Phase I trial of granulocyte macrophage colony-stimulating factor and interleukin-4 as combined immunotherapy for patients with cancer. Journal of immunotherapy (Hagerstown, Md.: 1997) 2003; 26:171-8.
38. Tate J, Olencki T, Finke J, Kottke-Marchant K, Rybicki L, Bukowski R. Phase I trial of simultaneously administered GM-CSF and IL-6 in patients with renal-cell carcinoma: Clinical and laboratory effects. Ann Oncol 2001; 12:655-9.
39. Olencki T, Finke J, Tubbs R, et al. Immunomodulatory effects of interleukin-2 and interleukin-4 in patients with malignancy. Journal of immunotherapy (Hagerstown, Md.: 1997) 1996; 19:69-80.
40. Reddy S, Harwood R, Moore D, Grimm E, Murray J, Vadhan-Raj S. Recombinant interleukin-2 in combination with recombinant interferon-gamma in patients with advanced malignancy: A phase I study. J Immunother 1997; 20:79-87.
41. Margolin K, Doroshow J, Akman S. Phase I study of interleukin-2 plus interferon-gamma. Journal of immunotherapy (Hagerstown, Md.: 1997) 1992; 11:50-5.
42. Negrier S, Escudier B, Lasset C. Recombinant human interleukin-2, recombinant human interferon alfa-2a, or both in metastatic renal-cell carcinoma. New Engl J Med 1998; 338:1272-8.
43. Sparano J, Fisher R, Sunderland M. Randomized phase III trial of treatment with high-dose interleukin-2 either alone or in combination with interferon alfa-2a in patients with advanced melanoma. Journal of clinical oncology: official journal of the American Society of Clinical Oncology 1993; 11:1969-77.
44. Eskander E D, Harvey H A, Givant E, Lipton A. Phase I study combining tumor necrosis factor with interferon-alpha and interleukin-2. Am J Clin Oncol 1997; 20:511-4.
45. Schmidinger M, Steger G, Wenzel C. Sequential administration of interferon-gamma, gm-csf, and interleukin-2 in patients with metastatic renal cell carcinoma: Results of a phase II trial. Journal of immunotherapy (Hagerstown, Md.: 1997) 2001; 24:257-62.
46. de Gast G, Klumpen H-J, Vyth-Dreese F, et al. Phase I Trial of Combined Immunotherapy with Subcutaneous Granulocyte Macrophage Colony-stimulating Factor, Low-Dose Interleukin 2, and Interferon a in Progressive Metastatic Melanoma and Renal Cell Carcinoma. Clinical cancer research: an official journal of the American Association for Cancer Research 2000; 6:1267-72.
47. Verra N, Jansen R, Groenewegen G ea. Immunotherapy with concurrent subcutaneous GM-CSF, low-dose IL2 and IFN-alpha in patients with progressive metastatic renal cell carcinoma. Br J Cancer 2003; 88:1346-51.
48. Valentine F. The transformation and proliferation of lymphocytes a review. In: Bloom B, Glade P, eds. In Vitro Methods in Cell-Mediated Immunity. New York: Academic Press; 1971:66-74.

What is claimed is:

1. A method for inducing a systemic immune response to a metastatic cancer in a subject, the method comprising administering supernatants from activated peripheral blood mononuclear cells (PBMCs) to the subject, wherein the supernatants are administered intratumorally and repeatedly.

2. The method of claim 1, wherein the metastatic cancer is a melanoma, breast cancer, renal cancer, or lung cancer.

3. The method of claim 1, wherein the supernatants are administered in a small volume.

4. The method of claim 3, wherein the small volume is about 0.05 to 3.0 milliliters per injected metastatic nodule.

5. The method of claim 1, wherein the peripheral blood mononuclear cells (PBMCs) are isolated from the subject and the supernatants are generated by contacting the PBMCs in vitro with an antigen recognized by the PBMCs, thereby activating the PBMCs.

6. The method of claim 5, wherein the antigen is a microbial antigen.

7. The method of claim 6, wherein the microbial antigen is tuberculin PPD skin test antigen, streptokinase for injection, Candida albicans skin test antigen, or tetanus toxoid suitable for booster use, or a similar microbial antigen to which the patient has immune responses.

8. The method of claim 1, wherein the supernatants are administered at a frequency of once or twice per week or once per 2 to 4 weeks until 4 months after all evidence of metastatic disease has disappeared.

9. The method of claim 1, wherein the subject in need thereof is a mammal.

10. The method of claim 9, wherein the mammal is a human.

11. The method of claim 1, further comprising administering Ipilimumab or Nivolumab to the subject to enhance immune responses in the subject, wherein the Ipilimumab or Nivolumab is administered concurrently with the supernatants or after administering the supernatants.

12. A method for inducing a systemic immune response to a metastatic cancer in a subject, the method comprising administering irradiated autologous tumor cells and supernatants from activated peripheral blood mononuclear cells (PBMCs) to the subject, wherein the irradiated autologous tumor cells and supernatants are administered intradermally and repeatedly.

13. The method of claim 12, wherein the metastatic cancer is a melanoma, breast cancer, renal cancer, or lung cancer.

14. The method of claim 12, wherein the supernatants are administered in a small volume.

15. The method of claim 14, wherein the small volume is about 0.05 to 3.0 milliliters per injection.

16. The method of claim 12, wherein the peripheral blood mononuclear cells (PBMCs) are isolated from the subject and the supernatants are generated by contacting the PBMCs in vitro with an antigen recognized by the PBMCs, thereby activating the PBMCs.

17. The method of claim 16, wherein the antigen is a microbial antigen.

18. The method of claim 12, wherein the irradiated autologous tumor cells and the supernatants are administered at a frequency of once per week or once per 2 to 4 weeks.

19. The method of claim 12, wherein the subject is a human.

20. The method of claim 12, further comprising administering Ipilimumab or Nivolumab to the subject to enhance immune responses in the subject, wherein the Ipilimumab or Nivolumab is administered concurrently with the irradiated autologous tumor cells and supernatants or after administering the irradiated autologous tumor cells and supernatants.
